SEQUENCE LISTING (1 sequence)

SEQ ID NO: 1 - DNA, 32 bp, Homo sapiens
ctaatacgactcactatagggctcgagcggcc

(19) United States
(12) Patent Application Publication
(10) Pub. No.: US 2014/0235466 A1
(43) Pub. Date: Aug. 21, 2014

(54) Tumor biomarkers for pancreatic marker

(71) Applicant: Manuel Gidekel, Santiago (CL)

(72) Inventor: Manuel Gidekel, Santiago (CL)

(73) Assignee: CTI

(21) Appl. No.: 13/815,320

(22) Filed: Feb. 21, 2013

(51) Int. Cl. C12Q 1/68

(52) U.S. Cl. CPC C12Q 1/6886
USPC 506/9

(57) ABSTRACT

This invention pertains generally to the fields of molecular biology and medical diagnosis. More particularly this invention provides novel biomarkers for the detection of pancreatic cancer comprising at least one messenger RNA and/or up-regulated peptide in pancreatic cancer. The present invention provides biomarkers capable of discriminating between non-neoplastic pancreatic tissues and ductal adenocarcinoma tissues and can be correlated with a probable diagnosis of pancreatic cancer as well as assessing the efficacy of ongoing therapies for this disease.

### FIELD OF THE INVENTION

This invention pertains generally to the fields of molecular biology and medical diagnosis. More particularly this invention provides novel biomarkers for the detection of pancreatic cancer comprising at least one messenger RNA and/or up-regulated peptide in pancreatic cancer. The present invention provides biomarkers capable of discriminating between non-neoplastic pancreatic tissues and ductal adenocarcinoma tissues and can be correlated with a probable diagnosis of pancreatic cancer as well as assessing the efficacy of ongoing therapies for this disease.

### BACKGROUND OF THE INVENTION

Pancreatic ductal adenocarcinoma (PDAC), also known as pancreatic cancer, has been identified as the fourth leading cause of cancer related death in the United States. This disease is more common in the elderly and only 20% of patients have localized tumors and potentially curable. Importantly, the survival rate in patients at 5 years is less than 5% [1], making the PDAC one of the deadliest solid tumors. The dreadful prognosis of PDAC is directly linked to the lack of methods for early diagnosis and effective antineoplasic treatments. The patient survival rate substantially improves if the tumor is diagnosed in early stages of the disease when they are amenable to surgical intervention. Recently it was demonstrated that the progression of PDAC, from the generation of the tumor cell of origin to the first appearance of metastatic clone, occurs in an average of 15 years, allowing a hypothetical time window for early diagnosis of the disease [2]. In this context it is important to identify molecular biomarkers to detect the tumor at an early stage and whose implementation can be transferable to routine clinical use. The antigen CA 19-9 is the only biomarker that has clinical utility, and it is used to monitor treatment response and detection of disease recurrence after treatment, but has little use as a method for early diagnosis of pancreatic cancer and is not specific to this cancer. Therefore, there is an urgent need to identify new strategies for early diagnosis of PDAC.

The methodology of suppression subtractive hybridization coupled with microarray allows the identification of specific genes of tumor cells whose expression is relatively low [3]. The genes identified by this methodology may have a role in the carcinogenesis process, and therefore have the potential to be used as therapeutic targets or tools for specific diagnosis of pancreatic cancer.

### SUMMARY OF THE INVENTION

The present invention provides methods for diagnosing the presence or absence of cancer in patients by detecting expression levels of one or more genes in tissues where expression of these genes is indicative of the presence of disease especially pancreatic cancer.

The present invention provides for the identification of genes whose gene products are specifically expressed in pancreatic ductal adenocarcinoma tissues and not in normal tissues or tissue adjacent to the tumor.

In one embodiment, the present invention provides for the measurement of parameters, alone or in combination, which may be correlated with a probable diagnosis of cancer.

In another embodiment, the present invention provides new therapeutic methods for the treatment of pancreatic cancer; particularly, the reduction of the expression or interfering with the biological function of some of the described genes, the monitoring of the progression of the disease and the a method to determine the efficacy of drugs used in treatment.

In yet another embodiment, the present invention provides a method to evaluate the efficacy of treatment of pancreatic cancer, comprising the steps of administering a pharmaceutical compound or specific surgical procedure, followed by comparing the gene expression profile in blood samples containing the patient's tumor cells with the profile obtained in healthy cells or tissue.

The expression of specific sets of genes in a cell is subject to spatial and temporal changes that allow the coordination of cellular processes and the maintenance of normal cell function. The pathological changes associated with the carcinogenic process and tumor progression are associated with alterations in the expression of genes that can influence cell behavior. These pathological changes can also be used as indicators or markers of the disease, thus allowing the potential diagnosis, monitoring and treatment of disease.

The present invention describes the changes in gene expression in pancreatic cancer compared to non-neoplastic tissue of the pancreas. Surgically removed tissues of malignant tumors of the pancreas were studied using a microarray platform coupled with the methodology of suppression subtractive hybridization to identify specific gene expression in tumor tissues. Gene expression profiling results are described in FIG. 1 and FIG. 2. Validation at protein level is shown FIG. 3-6.

The present invention provides methods to detect genes expressed specifically in tumor tissues compared to non-tumor tissue.

### DETAILED DESCRIPTION OF THE INVENTION

### Example 1

### Genes Differentially Expressed in Pancreatic Cancer

In order to identify differentially expressed genes in pancreatic cancer, we performed a paired analysis of 6 tumor tissues and their neoplastic tissues. We identified 150 genes up-regulated in tumor tissues by microarray analysis coupled with the strategy of suppression subtractive hybridization. FIG. 1 is a graph of type heatmap showing the expression profile of the 150 up-regulated genes identified. The names of the genes are described in FIG. 2. The measurement of these genes, alone or in combination, allow for discrimination between normal and tumor tissues. Genes that can be used for diagnosis of pancreatic cancer alone or in combination are shown in FIG. 2.

### Example 2

### Identification of Two Proteins as Pancreatic Cancer Biomarkers

At the protein level, by immunohistochemical staining, PLEKHM1 expression is up-regulated in PDAC tissues compared to normal tissues and non-neoplastic pancreatic (FIGS. 3A and 3B). PLEKHM1 is not expressed in ducts, acinar cells or islets of Langerhans in non-neoplastic tissue adjacent to tumor (FIG. 3A), however, we observed PLEKHM1 expression in 30% of pancreatic tumor tissues analyzed (5/16). This protein is specifically expressed by cells of epithelial origin expressing keratin 19 (FIG. 3C). In some cases, this protein is expressed by single cells in the invasive tumor front (FIG. 3D), suggesting a potential role in the process of tumor invasion and tumoral metastasis. PLEKHM1 positive cells are characterized by very disturbed morphologies and occur in pancreatic tumors of different histological type and different degrees of differentiation (FIGS. 4A, 4C and 4E). Through the use of Quantitative immunohistochemical analysis of tissue microarray (TMA) it was found that PLEKHM1 significantly expresses in tumor tissues compared with non-neoplastic pancreatic tissues (FIG. 5A).

To evaluate the specific expression of PLEKHM1 in pancreatic cancer, immunohistochemical stains were used on other organs using a TMA of normal tissues, where we observed that PLEKHM1 is not expressed in a normal pancreas, however, is expressed by prostate cell types (prostate), thymus (thymus), amygdala (tonsil), cervix (uterus cervix) and skin (skin), the latter being the organ where there is a greater expression of PLEKHM1 (FIG. 5B). There was not detected expression of PLEKHM1 in tissues of adrenal gland, bladder, bone marrow, eye, breast, cerebellum, cerebral cortex, Fallopian tube, stomach, esophagus, small intestine, colon, rectum, heart, kidney, liver, lung, ovary, parathyroid gland, pituitary gland, placenta, spleen, skeletal muscle, testis and thyroid gland.

On the other hand, another study findings was the identification of WNT9A expressed in pancreatic cancer specifically. WNT9A belongs to the family of WNT proteins and has been linked to the process of chondrogenesis and joint integrity in murine models [4], as well as morphogenesis and cell proliferation in liver of avian models [5]. In zebrafish has been linked to the development of the palate and lower jaw [6]. The WNT9A role in cancer has not been studied in depth.

WNT9A expression was analyzed in adenocarcinoma pancreatic ductal tissues by immunohistochemistry. WNT9A expresses in non-neoplastic pancreatic ductal cells with a granular staining pattern of supranuclear location, suggestive of location in the endoplasmic reticulum, however, this expression is low or absent in most small ducts (FIG. 6A), being the medium or bigger sized ducts the ones that expresses WNT9A in a more intense way (FIG. 6B). In adenocarcinoma ductal tissue, WNT9A is expressed in most tumor cells, frequently showing intense staining patterns (FIGS. 6C and 6D). WNT9A expression was evaluated by tissue microarray, following the same protocol used for PLEKHM1. In FIG. 6E we can see that WNT9A expression is significantly higher in adenocarcinoma ductal tissues by comparison to non-neoplastic tissues (FIG. 6E).

### BRIEF DESCRIPTION OF FIGURES

FIG. 1. Heatmap is a graph representing the 150 up-regulated genes in tissues of PDAC (adenocarcinoma ductal pancreatic) compared with non-neoplastic pancreatic tissues. The official symbol and the name of the 150 genes are shown in FIG. 2. Tumor tissues and non neoplastic can be identified based on gene expression profile. Red represents up-regulated genes and green represents down-regulated genes. Fold change indicates the number of changes.

FIG. 2. Is a table containing the 150 most statistically significant genes that are up-regulated in PDAC tissues compared to non neoplastic pancreatic tissue.

FIG. 3. PLEKHM1 expression is observed in regions that express positivity for keratin 19. A) shows the expression of PLEKHM1 in non-neoplastic pancreatic tissue, islets of Langerhans (red arrows), normal duct (black arrow). Acinar cells, islets and ducts do not express detectable levels of PLEKHM1. B) shows the expression PLEKHM1 in adenocarcinoma ductal pancreatic tissue. The picture shows tumoral cells expressing PLEKHM1 protein (brown staining). C) shows a three-color indirect immunofluorescence analysis on adenocarcinoma pancreatic tissue; PLEKHM1 (AlexaFluor 488, green), keratin 19 (AlexaFluor 568, red), DAPI nuclear staining (blue). In the analyzed tissues, the positive cells for keratin 19 express the protein PLEKHM1. D) shows invasive cells expressing PLEKHM1 (black arrows). E) Expression of keratin 19 for serial cutting of FIG. 3D.

FIG. 4. A) Expression of PLEKHM1 in adenosquamous pancreatic tumor. B) Expression of keratin 19 in a adenosquamous pancreatic tumor corresponding to a serial cutting of FIG. 4A. C) Expression of PLEKHM1 on adenocarcinoma ductal poorly differentiated. D) Expression of keratin 19 in poorly differentiated adenocarcinoma ductal, corresponding to a serial cutting of FIG. 4C. E) Expression of PLEKHM1 on adenocarcinoma ductal moderately differentiated. F) Expression of keratin 19 in adenocarcinoma ductal moderately differentiated corresponding to a serial cutting of FIG. 3E.

FIG. 5. A) T test two-tailed paired analysis of staining scores of PLEKHM1 for 30 cases of adenocarcinoma ductal and their respective neoplastic tissues. Dotted line represents the average score. B) No expression of PLEKHM1 in normal pancreas; PLEKHM1 expression in cell types of skin, prostate, thymus, tonsil, and cervix.

FIG. 6. A) null expression of WNT9A in a non-neoplastic tissue adjacent to a tumoral lesion. B) Medium and small ducts with WNT9A moderate expression at supranuclear level. C) WNT9A expression in adenocarcinoma ductal pancreatic tissues. D) WNT9A expression in adenocarsinoma ductal pancreatic tissues. E) Graphic of T test two tailed paired analysis of ACIS staining scores of WNT9A in 28 cases of pancreatic cancer and controls non-neoplastic from the same patients. Dotted line indicates the average staining score.

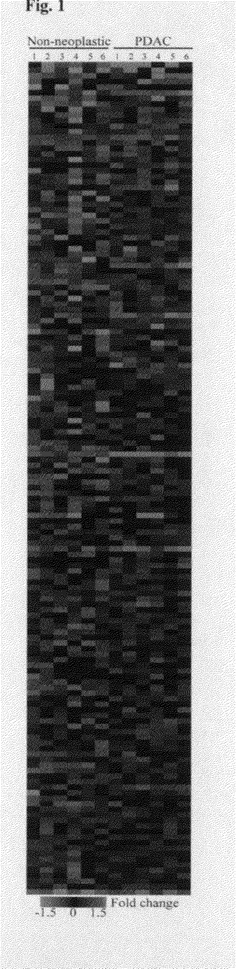

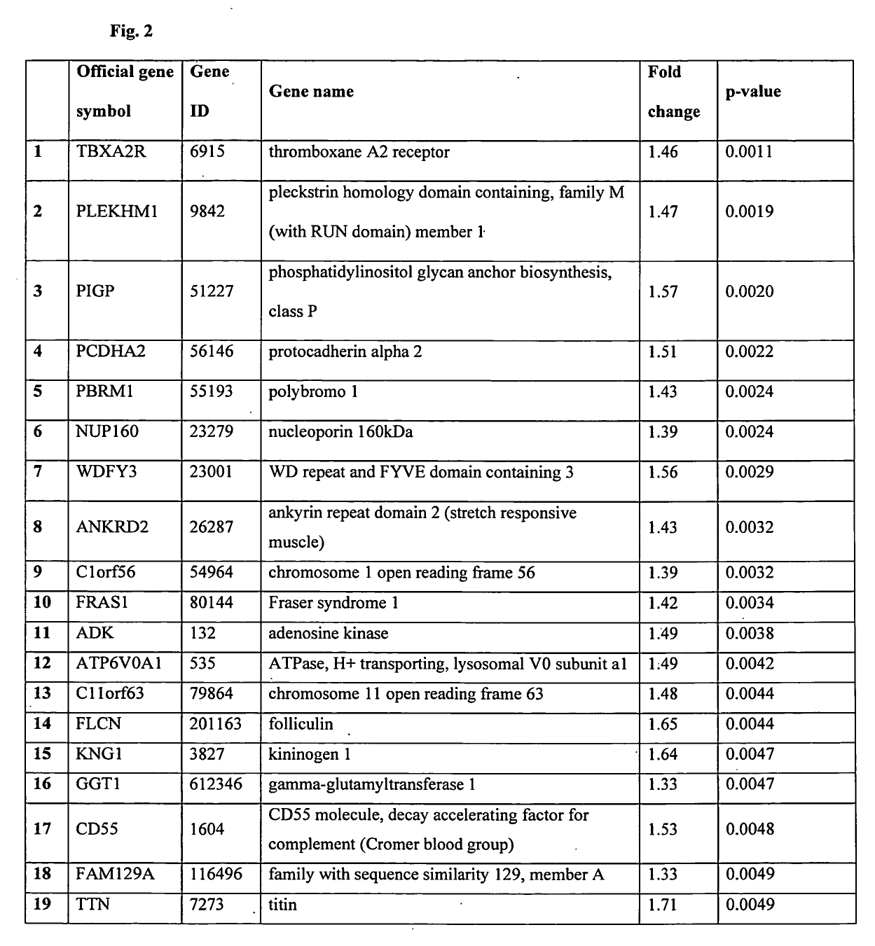

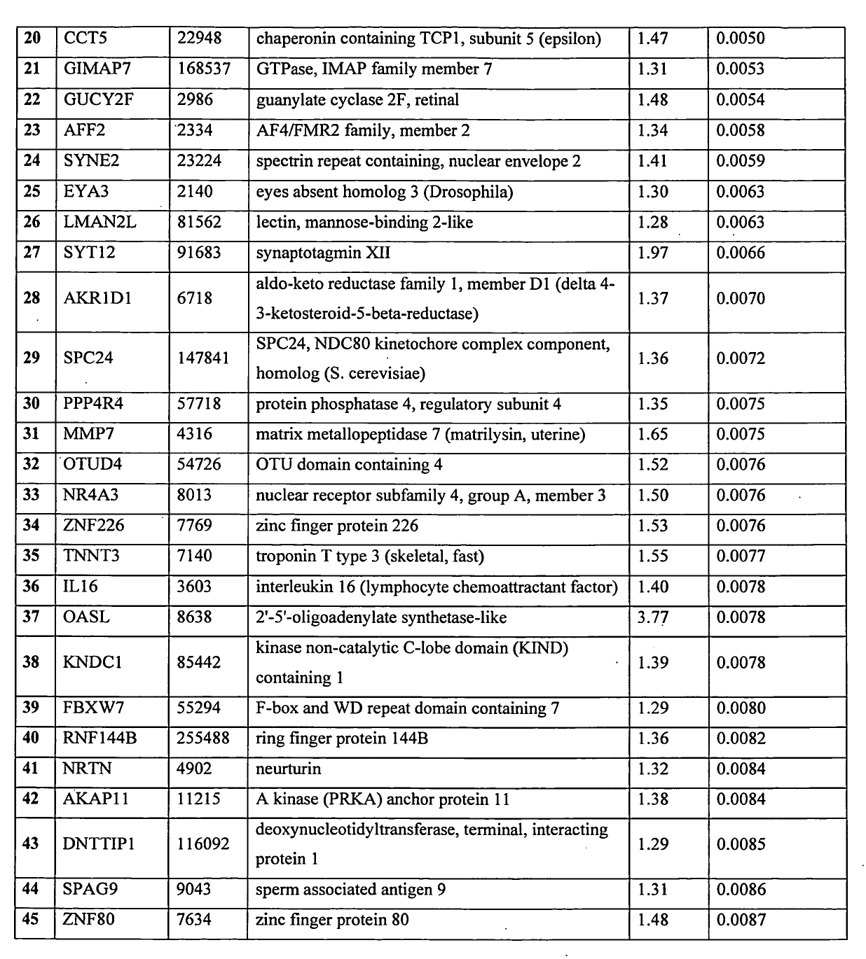

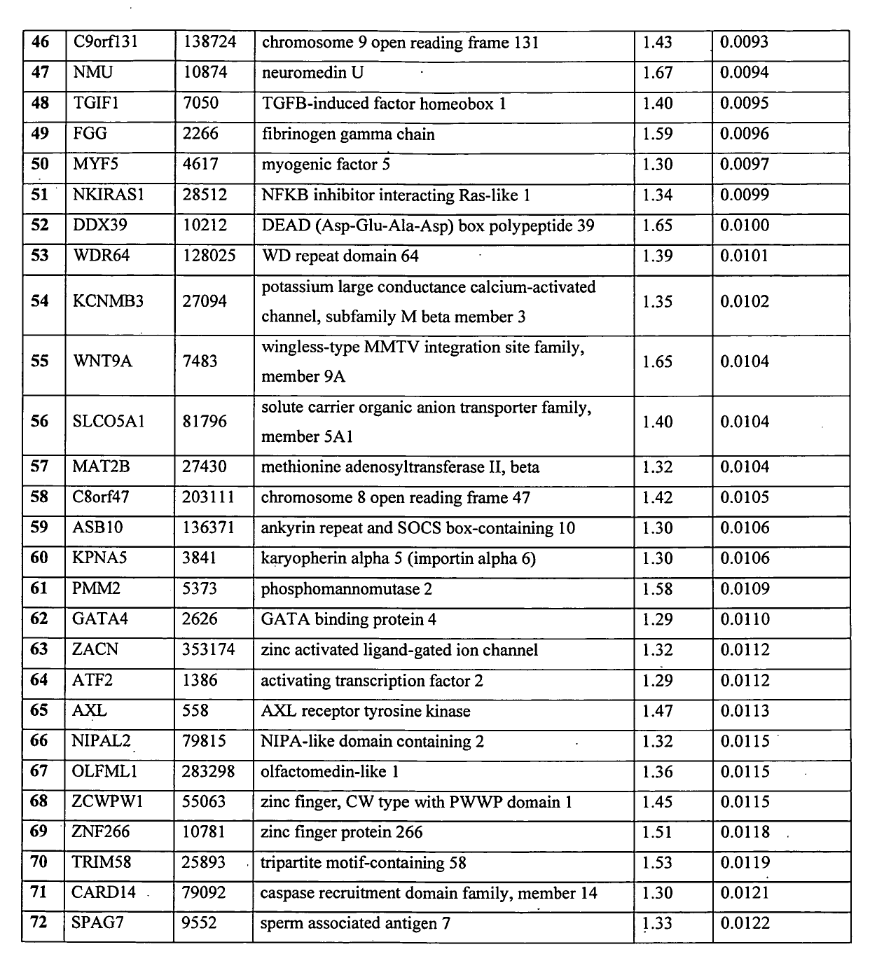

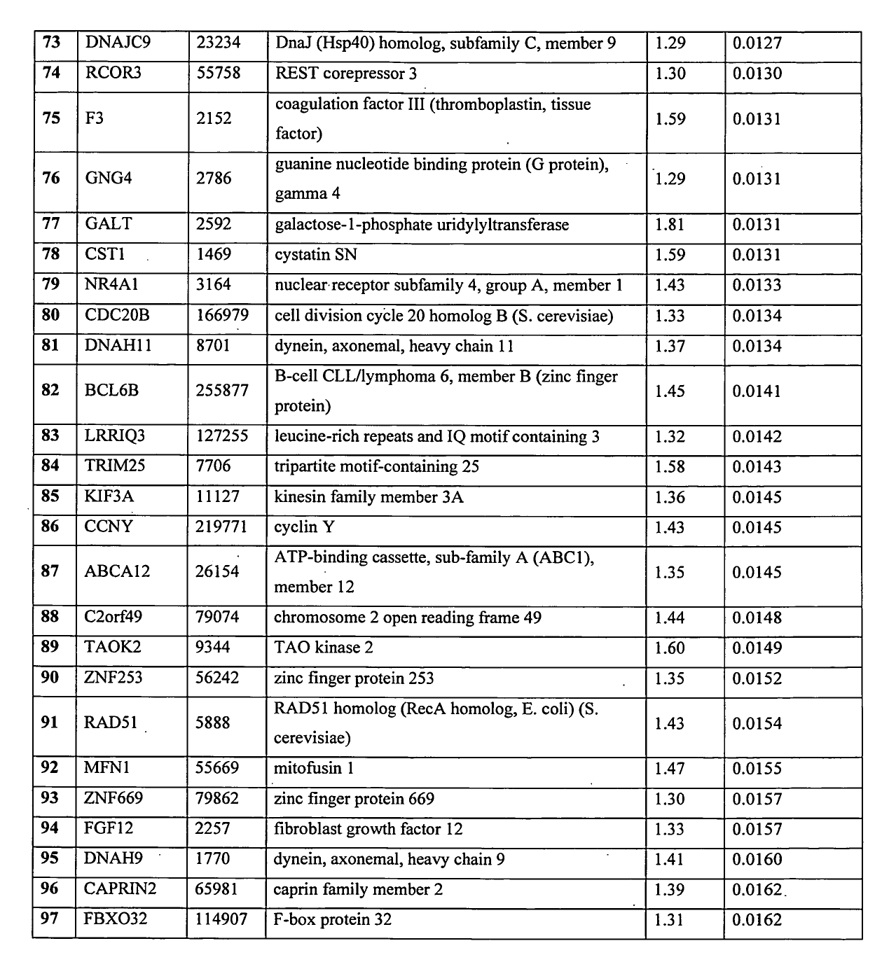

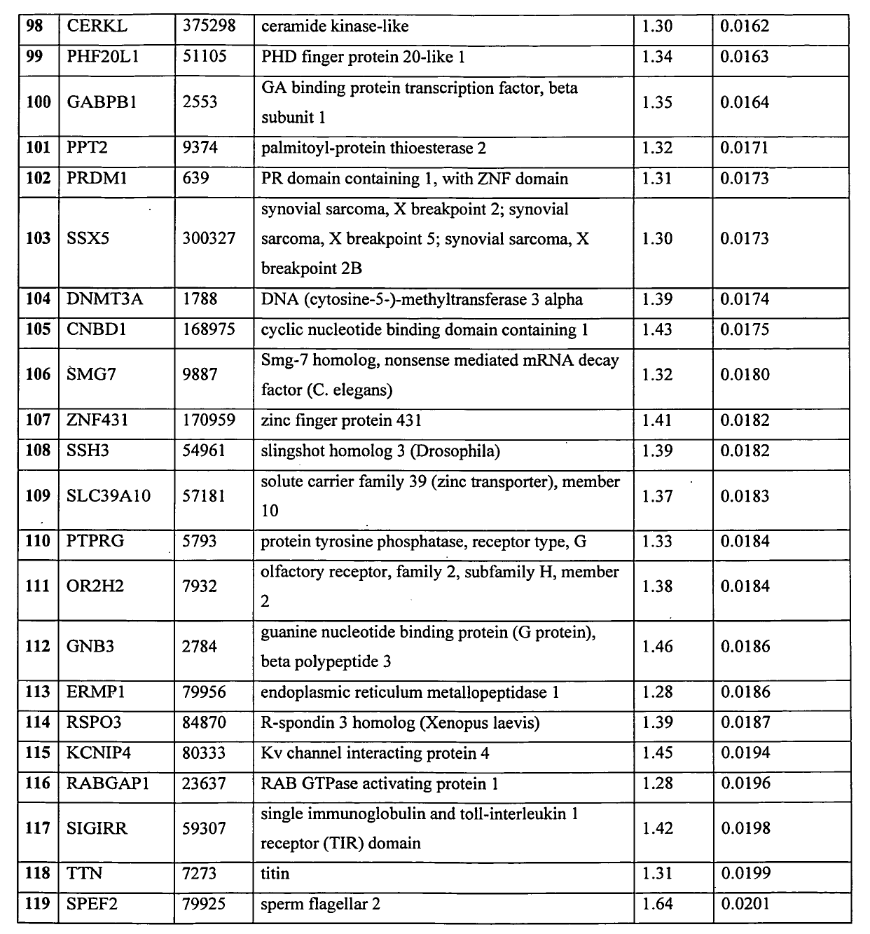

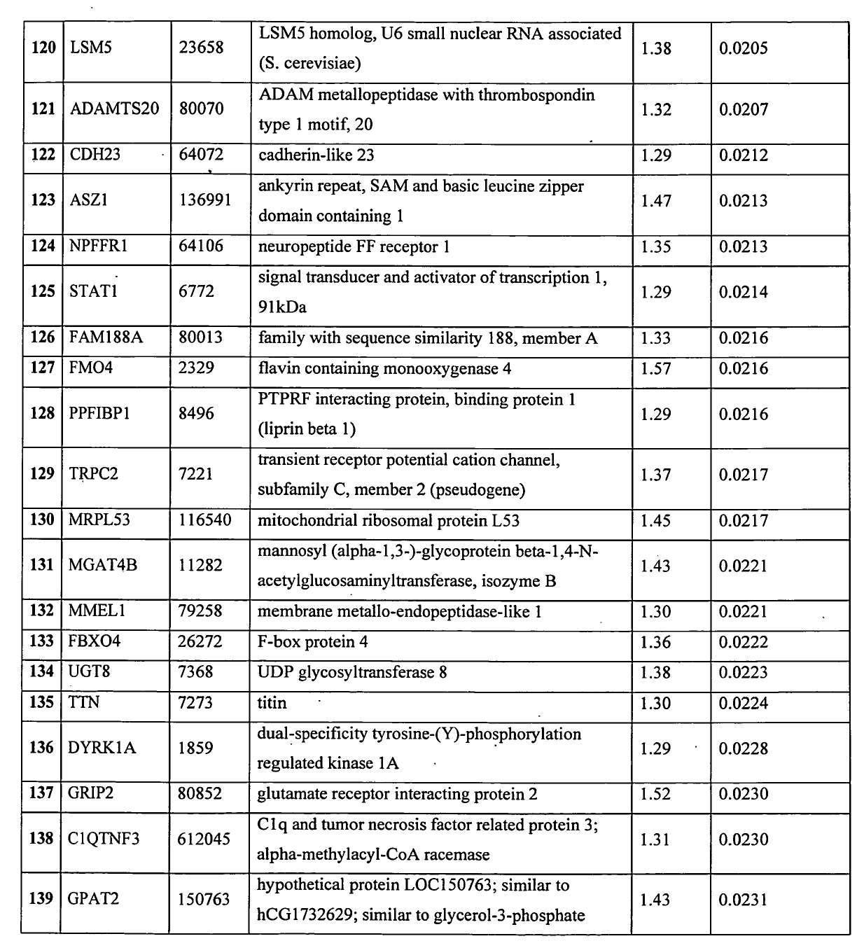

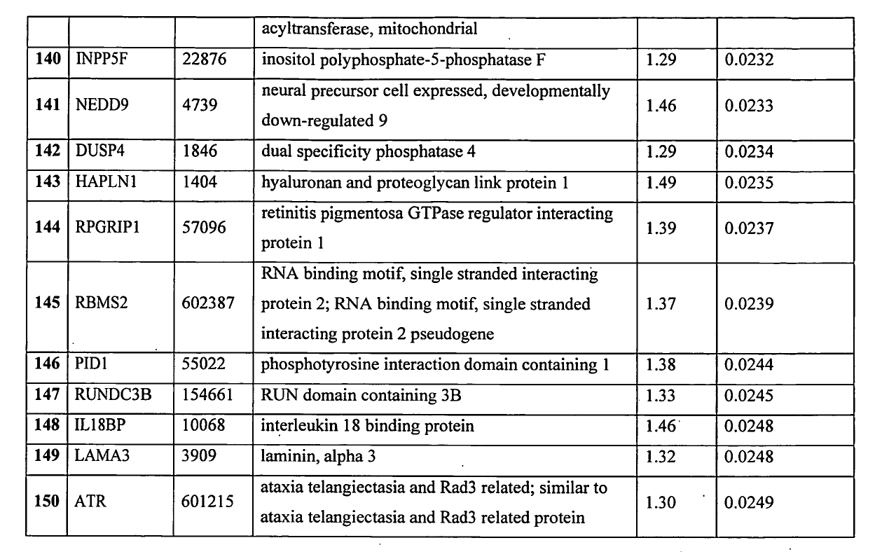

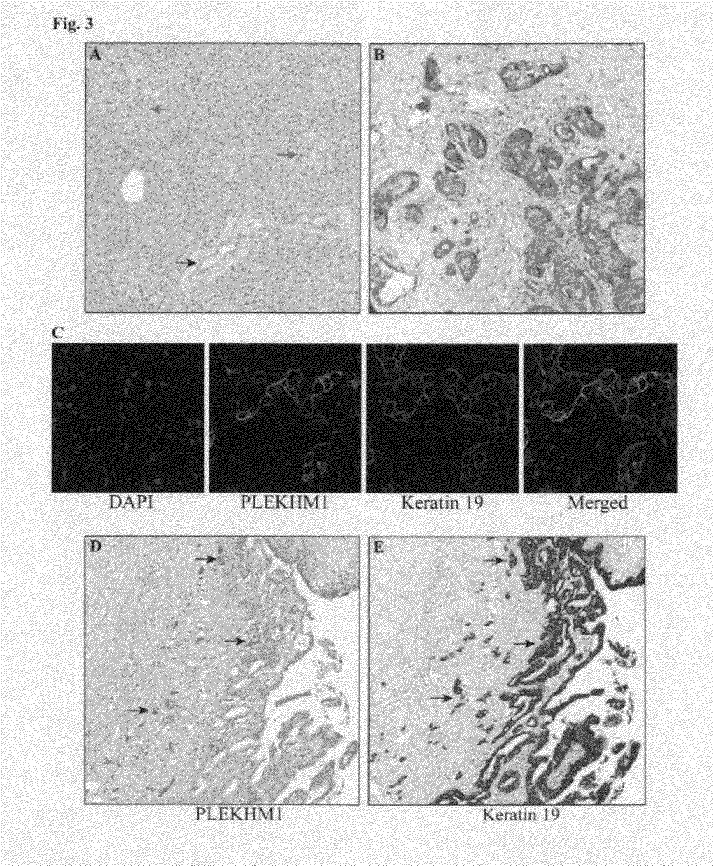

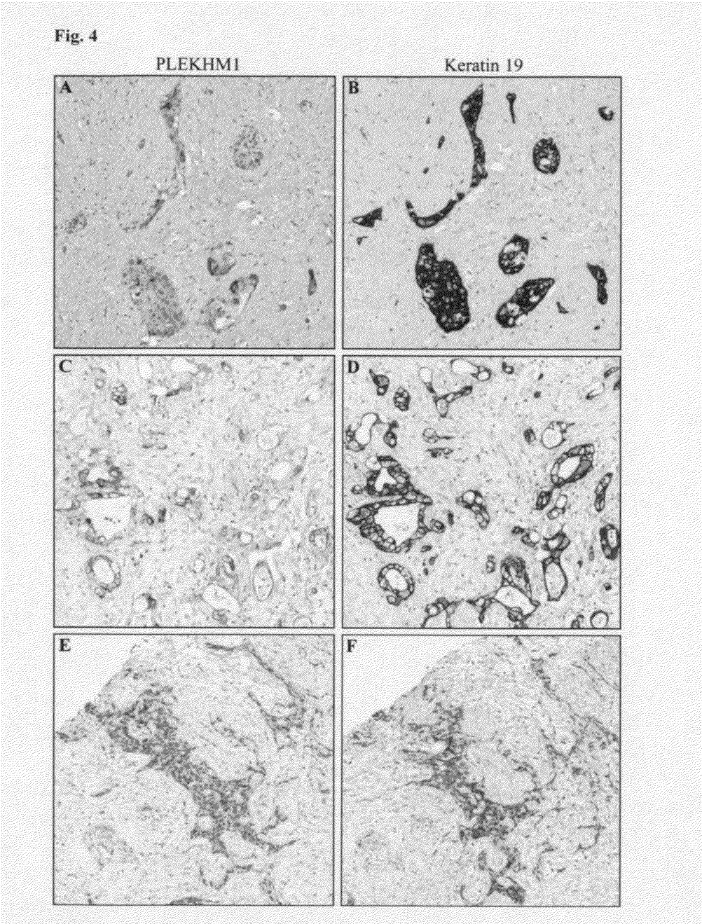

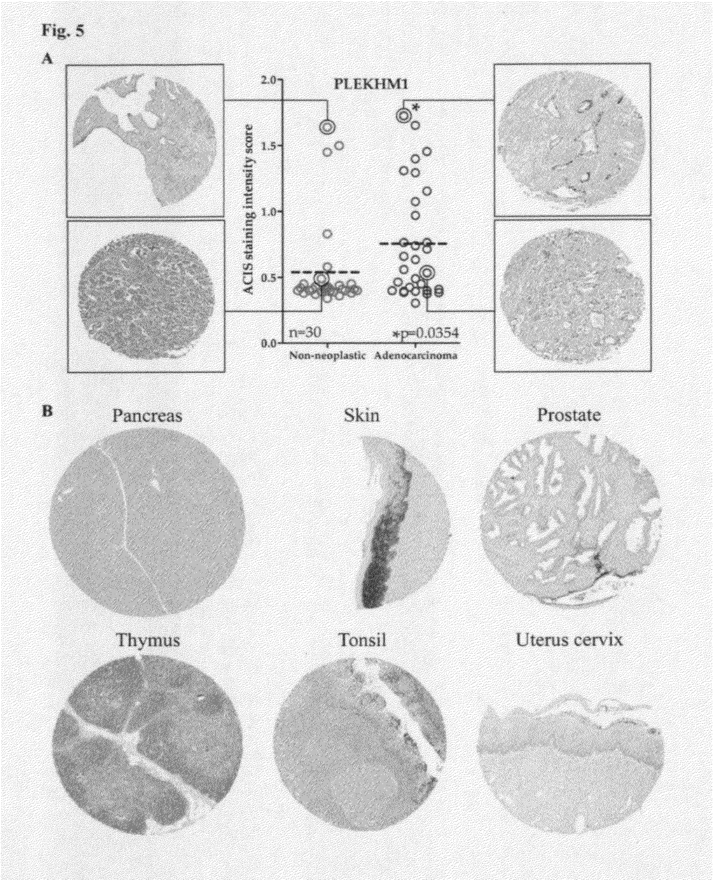

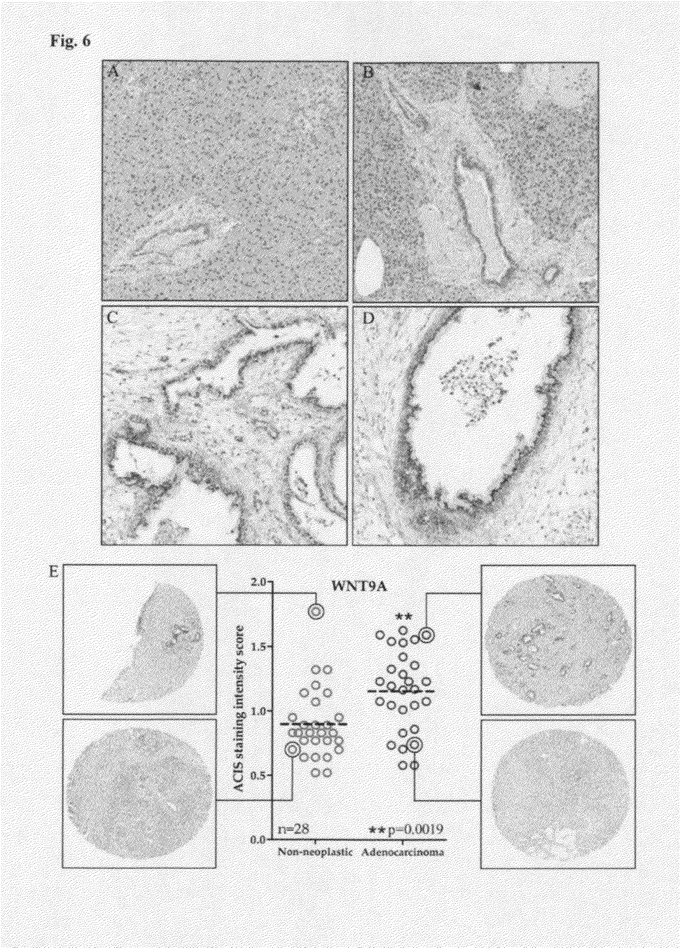

### MATERIALS AND METHODS

### Clinical Samples:

Adenocarcinoma ductal pancreatic tissues and non-neoplastic adjacent tissues to the tumoral lesion were collected rapidly after surgical resection and frozen at −80° C. until use. Tissue microarrays (TMA) for pancreatic cancer containing tissues of 30 patients, including 2 cores of tumoral tissue for each core of non-neoplastic tissue from the same patient (Array AccuMax A307). The analysis of expression in normal tissues was performed using a TMA Pantomic Normal Tissues MN0661, built with 33 normal tissues in duplicate (Pantomics Inc.).

### Suppressive Subtractive Hybridization

Total RNA was extracted from PDAC tissues using Trizol reagent (Invitrogen Corp., Carlsbad, Calif., USA) followed by purification using RNeasy mini kit columns (Qiagen, Hilden, Germany) according to the manufacturer's instructions. Human Universal Reference RNA and Human Pancreas Total RNA were purchased from Clontech (Palo Alto, Calif., USA). RNA integrity was assessed by denaturing agar and ultraviolet spectrophotometer. Total RNA from PDAC and non-neoplastic tissues were used as tester and commercial Human Pancreas Total RNA served as driver. First strand cDNA was synthesized from 1 ug of total RNA using Super SMART PCR cDNA Synthesis Kit (Clontech, Palo Alto, Calif., USA) and SuperScript III Reverse Transcriptase (Invitrogen, Carlsbad, Calif., USA) following the supplier's protocol. SSH procedures were essentially the same described in the manufacturer's instructions except for the use of a modified nested PCR Primer 5 2R 5′ CTAATACGACTCACTATAGGGCTCGAGCGGCC-3′ in the secondary PCR, which includes a T7 promoter site to carry out In Vitro transcription of the subtractive amplicon. After secondary PCR, subtractive cDNA was purified using E.Z.N.A Cycle Pure Kit (Omega Bio-Tek, Norcross, Ga., USA). For in vitro transcription, 300 ng of the purified cDNA subtractive cDNA was used as template. The newly aRNA was synthesized and labeled with aminoallyl-UTP and AlexaFluor 647 (Invitrogen, Carlsbad, Calif., USA) using the SuperScript Indirect RNA Amplification System (Invitrogen, Carlsbad, Calif., USA). aRNA for the Direct strategy was amplified and labeled directly from 1 ug of total RNA using the same system described above. Labeled aRNA from Direct and SSH-strategy were employed for hybridization on 48.5K Exonic Evidence Based Oligonucleotide (HEEBO) arrays, Purchased from Microarray Inc. (Nashville, Tenn., USA). Prior to hybridization, slides were pre-blocked with 5×SSC, 0.1% BSA and 0.1% SDS. Fluorescent-labeled probe were mixed with 1× hybridization solution (5×SSC, 50% formamide; 0.1% SDS, and 0.01% salmon sperm DNA) and heated at 95° C. for 2 min. Samples were hybridized on microarray slides for 16 hrs at 42° C. Slides were scanned using a ScanArray Gx (Perkin Elmer, Waltham, Mass., USA).

### Microarray Data Analysis

Microarray signal intensity was evaluated by SpotReader Software (Niles Scientific, Portola Valley, Calif., USA). Normalization was performed in R statistical environment using Limma package (www.r-proyect.org). Raw data from individual arrays were processed using standard and normexp background correction [7] and printtiploess normalization [8]. Global scale normalization function using median absolute deviation was used for normalization between arrays [9]. Heatmaps were constructed using MeV software [10].

### Antibodies and Immunohistochemical Analysis:

The following antibodies were used in this study for the validation of the corresponding gene candidate: PLEKHM1 (Human Atlas Protein HPA021558, dilution 1:75); WNT9A (Human Atlas Protein HPA011223; 1:25). Histological sections of 4 um thick sections were mounted on Super Frost slides, incubated at 65° C. for 60 min, dewaxed, blocked with 1% hydrogen peroxide for 10 min and rehydrated by successive incubations in graded alcohols. Antigen retrieval was performed by incubating the slides in a buffer of Tris/EDTA pH 9.0 (10 mM Tris, 1 mM EDTA) and heated in a microwave at 750 W for 10 min. Subsequently, the samples were incubated in serum 1% in TBS buffer to block nonspecific staining. The presence of the antigen was evaluated by incubating the samples for 45 min with primary antibody followed by detection with secondary antibody conjugated to peroxidase complex system EnVision poly-HRP (DAKO) for 45 min. The samples were then incubated with DAB+chromogen (Dako) for 15 minutes for color development and finally contrasted with hematoxylin.

The analysis of immunohistochemical staining (IHC) was performed using the automated ACIS III (DAKO) to digitize and quantify IHC staining. The ACIS III software can recognize separately brown pixels (positive staining) and blue pixels (hematoxylin counterstain). For analysis of the staining TMAs generate a score depending on the intensity of staining and the percentage of cells showing immunoreactivity within the core.

### REFERENCES

1. Hidalgo M: Pancreatic cancer. N Eng J Med 2010, 362:1605-1617.
2. Yachida S, Jones S, Bozic I, Antal T, Leary R, Fu B, Kamiyama M, Hruban R H, Eshleman J R, Nowak M A, et al: Distant metastases late Occurs During The genetic evolution of pancreatic cancer. Nature 2010, 467:1114-1117.
3. Liu B H, Goh C H, Ooi L L, Hui K M: Identification of unique and common low abundance transcripts Tumour-specific suppression subtractive hybridization by oligonucleotide probe and array analysis. Oncogene 2008, 27:4128-4136.
4. Spater D, Hill T P, O'Sullivan R J, Gruber M, Conner D A, Hartmann C: Wnt9a signaling is required for joint integrity and regulation of lhh DURING chondrogenesis. Development 2006, 133:3039-3049.
5. Matsumoto K, Miki R, Nakayama M, Tatsumi N, Yokouchi Y: Wnt9a secreted from the walls of hepatic sinusoids is essential for morphogenesis, proliferation, and glycogen Accumulation of chick hepatic epithelium. Dev Biol 2008, 319:234-247.
6. E Curtin, Hickey G, Kamel G, Davidson A J, Liao E C: wnt9a Zebrafish Expressed in pharyngeal ectoderm is and is required for palate and lower jaw development. Mech Dev 2011, 128:104-115.
7. Ritchie M E, Silver J, Oshlack A, Holmes M, Diyagama D, Holloway A, Smyth G K: A comparison of background correction methods for two-color microarrays. Bioinformatics 2007, 23:2700-2707.
8. Smyth G K, Speed T: Normalization of cDNA microarray data. Methods 2003, 31:265-273.
9. Yang Y H, Dudoit S, Luu P, Lin D M, Peng V, Ngai J, Speed T P: Normalization for cDNA microarray data: a robust composite method single and multiple slide Addressing Systematic variation. Nucleic Acids Res 2002, 30: e15.
10. Saeed A I, Bhagabati N K Braisted J C, Liang W, Sharov V, Howe E A Li J Thiagarajan M White J A, Quackenbush J: T M4 microarray software suite. Methods in enzymology 2006, 411:134-193.

1. A method for analyzing a biological sample from a human being for the presence of differentially expressed genes in pancreatic cancer comprising the steps of:
(a) obtaining the biological sample from the human being in the form of pancreatic tissue;
(b) analyzing the sample for the presence of genes known to be expressed specifically in pancreatic tumor tissues;
(c) comparing the analysis of step (b) with an analysis of known non-tumor tissue to determine the presence of genes known to be expressed specifically in pancreatic tumor tissues; wherein said analysis of step (b) is further comprised of the steps:
(i) performing a paired analysis of 6 tumor tissues and their neoplastic tissues;
(ii) identifying 150 genes up-regulated in tumor tissues by microarray analysis coupled with the strategy of suppression subtractive hybridization.

2. A method for analyzing a biological sample from a human being for the presence of a protein expressed in pancreatic cancer tissue comprising the steps of:
(a) obtaining the biological sample from the human being;
(b) analyzing the sample by immunohistochemical staining for the presence of protein PLEKHM1 known to be expressed in pancreatic cancer tissue;
(c) comparing the analysis of step (b) with an analysis of known non-tumor tissue to determine the presence of protein PLEKHM1 known to be expressed in pancreatic cancer tissue.

3. The method of claim 1 wherein the analysis of pancreatic tissue samples from a human being are correlated with a diagnosis of pancreatic cancer.

4. The method of claim 2 wherein the analysis of pancreatic tissue samples from a human being are correlated with a diagnosis of pancreatic cancer.

5. The method of claim 1 wherein the analysis of pancreatic tissue samples from a human being are used to monitor the progression of pancreatic cancer in a human being.

6. The method of claim 2 wherein the analysis of pancreatic tissue samples from a human being are used to monitor the progression of pancreatic cancer in a human being.

7. The method of claim 1 wherein the analysis of pancreatic tissue samples from a human being are used to determine the efficacy of drugs used in the treatment of pancreatic cancer.

8. The method of claim 2 wherein the analysis of pancreatic tissue samples from a human being are used to determine the efficacy of drugs used in the treatment of pancreatic cancer.
